(19) United States
(12) Patent Application Publication
(10) Pub. No.: US 2006/0030003 A1
(43) Pub. Date: Feb. 9, 2006

(54) Composition and method for introduction of RNA interference sequences into targeted cells and tissues

(76) Inventor: Michael R. Simon, Ann Arbor, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/186,609

(22) Filed: Jul. 21, 2005

Related U.S. Application Data
(63) Continuation-in-part of application No. 11/126,562, filed on May 11, 2005.
(60) Provisional application No. 60/570,200, filed on May 12, 2004.
(60) Provisional application No. 60/606,017, filed on Aug. 31, 2004.
(60) Provisional application No. 60/625,276, filed on Nov. 5, 2004.
(60) Provisional application No. 60/642,319, filed on Jan. 7, 2005.
(60) Provisional application No. 60/665,958, filed on Mar. 29, 2005.

(51) Int. Cl. C07H 21/04; C12P 21/06; C12N 5/06; C07K 16/46

(52) U.S. Cl. 435/69.1; 530/391.1; 435/320.1; 435/329; 536/23.53

(57) ABSTRACT

A composition and method are provided by which double-stranded RNA containing small interfering RNA nucleotide sequences is introduced into specific cells and tissues for the purpose of inhibiting gene expression and protein production in those cells and tissues. Intracellular introduction of the small interfering RNA nucleotide sequences is accomplished by the internalization of a target cell specific ligand bonded to a RNA binding protein to which a double-stranded RNA containing a small interfering RNA nucleotide sequence is adsorbed. The ligand is specific to a unique target cell surface antigen. The ligand is either spontaneously internalized after binding to the cell surface antigen. If the unique cell surface antigen is not naturally internalized after binding to its ligand, internalization is promoted by the incorporation of an arginine-rich peptide, or other membrane permeable peptide, into the structure of the ligand or RNA binding protein or attachment of such a peptide to the ligand or RNA binding protein. The composition and method are practiced in whole living mammals, as well as cells living in tissue culture.

### RELATED APPLICATIONS

This application is a continuation-in-part of U.S. patent application Ser. No. 11/126,562 filed May 11, 2005, which claims priority of U.S. Provisional Patent Application Ser. No. 60/570,200 filed May 12, 2004; Ser. No. 60/606,017 filed Aug. 31, 2004; Ser. No. 60/625,276 filed Nov. 5, 2004; Ser. No. 60/642,319 filed Jan. 7, 2005; and Ser. No. 60/665,958 filed Mar. 29, 2005.

### FIELD OF THE INVENTION

The present invention relates in general to gene product suppression and in particular to gene product suppression through delivery of double-stranded RNA or small hairpin RNA targeting a particular protein within a subject.

### BACKGROUND OF THE INVENTION

RNA interference (RNAi) is the process whereby messenger RNA (mRNA) is degraded by small interfering RNA (siRNA) derived from double-stranded RNA (dsRNA) containing an identical or very similar nucleotide sequence to that of the target gene. (Waterhouse 2001; Hutvagner and Zamore 2002a and 2002b; Lewis 20020132788; Lewis 20030092180; Kreutzer 20040038921; Scaringe 20040058886). This process prevents the production of the protein encoded by the targeted gene. Allele-specific silencing of dominant disease genes can be accomplished (Miller 2003).

The benefits of preventing specific protein production in mammals include the ability to treat disease caused by such proteins. Such diseases include those that are caused directly by such a protein such as multiple myeloma which is caused by harmful concentrations of a monoclonal immunoglobulin as well as diseases in which the protein plays a contributory role such as the effects of inflammatory cytokines in asthma.

Introduction of dsRNA into mammalian cells induces an interferon response which causes a global inhibition of protein synthesis and cell death. However, dsRNA several hundred base pairs in length have been demonstrated to be able to induce specific gene silencing following cellular introduction by a DNA plasmid (Diallo M et al. Oligonucleotides 2003).

### SUMMARY OF THE INVENTION

A composition includes long or short double-stranded RNA (dsRNA) adsorbed to an RNA binding protein illustratively including a histone, RDE-4 protein, or protamine, the RNA binding protein being covalently bound to a cell surface receptor specific ligand or integrated into the ligand such that the RNA binding protein and ligand create a single protein. The dsRNA is then hydrolyzed by Dicer, an RNAse III-like ribonuclease, thereby releasing siRNA that silences the target gene. The cell surface receptor specific ligand is a natural peptide, natural protein, or a protein such as an immunoglobulin fragment that is engineered to bind to the targeted receptor. The internalization of the ligand-bound dsRNA is optionally facilitated by the incorporation of a membrane-permeable arginine-rich peptide, pentratin, transportan, or transportan deletion analog into the ligand or attachment of such a peptide to the ligand.

### DETAILED DESCRIPTION OF THE INVENTION

The present invention has utility in suppression of deleterious gene expression products. Production of specific proteins is associated with allergic reactions, transplant organ rejection, cancer, and IgA neuropathy, to name but a few of the medical conditions a subject may suffer. Additionally, according to the present invention, it is appreciated that specific animal proteins are also suppressed in foodstuffs such as cow's milk, through the treatment of the animal. Inventive compositions include one of a long or short dsRNA, or short hairpin RNA (shRNA) that is adsorbed to a RNA binding protein that is covalently bound to a cell surface receptor specific ligand or integrated into the ligand such that the RNA binding protein and ligand create a single protein. The ligand is targeted to a specific tissue and/or cell type upon delivery to a subject. In designing a ligand coupled dsRNA or shRNA binding protein, a target tissue and/or cell is selected, and the targeted cell type is analyzed for receptors that internalize ligands following receptor-ligand binding. It is appreciated that the present invention is also operative in suppressing genes within a cell growing in vitro and particularly well suited for limiting contaminants in recombinant protein manufacture.

Cell specific antigens which are not naturally internalized are operative herein by incorporating an arginine-rich peptide within the ligand, an arginine-rich peptide attached to the cell surface receptor specific ligand, as detailed in U.S. Pat. No. 6,692,935 B1 or U.S. Pat. No. 6,294,353 B1. An arginine-rich peptide causes cellular internalization of a coupled molecule upon contact of the arginine-rich peptide with the cell membrane. Pentratin and transportan are appreciated to also be operative as vectors to induce cellular internalization of a coupled molecule through attachment to the cell surface receptor specific ligand as detailed in U.S. Pat. No. 6,692,935 B1 or U.S. Pat. No. 6,294,353 B1.

A cell surface receptor specific ligand as used herein is defined as a molecule that binds to a receptor or cell surface antigen. A ligand is then coupled to an appropriate dsRNA binding protein. The ligand is a natural- or engineered-peptide or protein, such as is commercially available (Antibodies by Design, MorphoSys, Martinsried, Germany) (U.S. Pat. No. 5,514,548; U.S. Pat. No. 6,653,068 B2; U.S. Pat. No. 6,667,150 B1; U.S. Pat. No. 6,696,245; U.S. Pat. No. 6,753,136 B1; U.S. 2004/017291 A1). Another specific engineered peptide that is commercially available is the camelid single heavy chain variable domain (Nanobodies, Ablynx, Nev.; Zwijnaarde, Belgium); such a variable domain heavy chain antibody fragment is humanized and the antigen specificity thereof is generated from a phage display library from an immunized animal (van Koningbruggen et al. 2003) or a nucleic acid sequence expression library from non-immunized animals, as detailed in EP 0 584 421 A1 or U.S. Pat. No. 6,399,763.

If the engineered ligand is an immunoglobulin, the carboxy terminus of the molecule is at the variable end of the protein, and the amino terminus is available for covalently binding to the RNA binding protein to which the dsRNA is adsorbed. Because of the relatively large size of immunoglobulin molecules, preferably a Fab fragment is used as the ligand rather than the entire immunoglobin. More preferably, a (Fab′)² fragment is provided that allows for divalent binding as would occur with the entire immunoglobin without the encumbrance of the Fc component. Bridging of cell surface receptors by a divalent (Fab′)² fragment facilitates activation of the signaling pathway and subsequent internalization of the receptor-ligand combination in some internalization processes.

The functional RNA interference activity of interfering RNA transported into target cells while adsorbed to a fusion protein containing protamine as the RNA bonding protein and a Fab fragment specific for the HIV envelope protein gp160 has been demonstrated (Song et al. 2005). Similarly, functional RNA interference activity of interfering RNA transported into target cells as a cargo molecule attached to HIV-1 transactivator of transcription (TAT) peptide₄₇₋₅₇ has been demonstrated (Chiu Y-L et al. 2004). The functional RNA interference activity of interfering RNA transported into target cells as a cargo molecule attached to pentratin has also been demonstrated (Muratovska and Eccles 2004).

The dsRNA or shRNA oligonucleotide mediating RNA interference is delivered into the cell by internalization of the receptor.

In the event a targeted cell receptor is a unique receptor that is not naturally internalized, that receptor is nonetheless suitable as a target by incorporating an internalization moiety such as an arginine-rich membrane permeable peptide within the ligand or attaching to the ligand such as an arginine-rich membrane permeable peptide, pentratin, or transportan as detailed in U.S. Pat. No. 6,692,935 B1 or U.S. Pat. No. 6,294,353 B1. This is readily accomplished using established plasmid technology (Caron et al. 2004; He et al. 2004). Alternatively, the use of MorphoSys' commercial trinucleotide mutagenesis technology allows the synthesis of a membrane-permeable arginine-rich peptide at a single position of the variable region, as detailed in U.S. Pat. No. 6,692,935 B1 or U.S. Pat. No. 6,294,353 B1. The MorphoSys system joins an antigen-non-specific Fab fragment containing a membrane-permeable arginine-rich peptide to an engineered Fab fragment with a variable region specific for the cell surface receptor in order to provide for the cell specific targeting of the dsRNA. These Fab fragments are joined by a helix-turn-helix region. Alternatively, the membrane-permeable arginine-rich peptide is incorporated into the antigen-specific Fab immunoglobulin fragment to yield a bivalent antigen specific molecule produced (Anderson D C 1993). The membrane-permeable arginine-rich peptide is optionally also attached to another portion of the immunoglobulin molecule (Mie M et al. 2003; U.S. Pat. No. 6,692,935 B1; U.S. Pat. No. 6,294,353 B1). Similarly, pentratin or transportan is attached to or incorporated within any ligand portion of the molecule with the proviso that ligand-receptor binding is maintained. In each situation, the ligand containing the membrane-permeable arginine-rich peptide, pentratin, or transportan serves to carry the dsRNA into the targeted cell.

Arginine-rich peptides which are internalized after contact with the cell membrane have been shown to transport covalently coupled proteins into cells (Peitz M et al. 2002, Jo et al. 2001). Examples of such internalization moieties illustratively include: membrane-permeable arginine-rich peptides, pentratin, transportan and its deletion analogs.

| | | |
|---|---|---|
| GRKKRRQRRRPPQ | (TAT 48-60) | (SEQ ID NO.1) |
| GRRRRRRRRRPPQ | (R9-TAT) | (SEQ ID NO.2) |
| TRQARRNRRRRWRERQR | (HIV-1 Rev 34-50) | (SEQ ID NO.3) |
| RRRRNRTRRNRRRVR | (FHV coat 35-49) | (SEQ ID NO.4) |
| KMTRAQRRAAARRNRWTAR | (BMVgag7-25) | (SEQ ID NO.5) |
| TRRQRTRRARRNR | (HTLV-II Rex 4-16) | (SEQ ID NO.6) |

Other membrane-permeable peptides are pentratin and transportan,

| | |
|---|---|
| (Atennapedia 43-58 - pentratin) | |
| RQIKIWFQNRRMKWKK | (SEQ ID NO.7) |
| (transportan) (Muratovska and | |
| Eccles 2004). | |
| LIKKALAALAKLNIKLLYGASNLTWG | (SEQ ID NO.8) |

Alternative amino acid composition for transportan and its deletion analogs which maintain membrane transduction properties (Soomets et al. 2000):

| | |
|---|---|
| (transportan) | |
| GWTLNSAGYLLGKINLKALAALAKKIL | (SEQ ID NO.9) |
| (transportan7) | |
| LNSAGYLLGKINLKALAALAKKIL | (SEQ ID NO.10) |
| (transportan9) | |
| GWTLNSAGYLLGKLKALAALAKKIL | (SEQ ID NO.11) |
| (transportan10) | |
| AGYLLGKINLKALAALAKKIL | (SEQ ID NO.12) |
| (transportan12) | |
| LNSAGYLLGKLKALAALAKKIL | (SEQ ID NO.13) |
| (transportan14) | |
| AGYLLGKLKALAALAKKIL | (SEQ ID NO.14) |

TAT=HIV-1 transactivator of transcription; FHV=flock house virus; BMV=brome mosaic virus.

Preferably, the internalization moiety is coupled to or incorporated into an immunoglobulin ligand which is bonded to an inventive dsRNA binding protein, or short hairpin RNA binding protein, the adsorbed dsRNA or shRNA serving as a substrates for enzymatic production of siRNA.

In another embodiment the internalization moiety is coupled to, or incorporated into, the RNA binding protein which is coupled to the ligand.

Receptor-binding immunoglobulins are obtained using hybridoma technology. Fab and (Fab′)² fragments are prepared from such immunoglobulins by papain and pepsin hydrolysis, respectively (Stura et al. 1993). The resulting molecules are purified using standard biochemical methods.

DsRNA with siRNA sequences that are complementary to the nucleotide sequence of the target gene are prepared. The siRNA nucleotide sequence is obtained from the siRNA Selection Program, Whitehead Institute for Biomedical Research, Massachusetts Institute of Technology, Cambridge, Mass. (http://jura.wi.mit.edu) after supplying the Accession Number or GI number from the National Center for Biotechnology Information website (www.ncbi.nlm.nih.gov). The Genome Database (www.gdb.org) provides the nucleic acid sequence link which is used as the National Center for Biotechnology Information accession number. Preparation of RNA to order is commercially available (Ambion Inc., Austin, Tex.; GenoMechanix, LLC, Gainesville, Fla.; and others). Determination of the appropriate sequences would be accomplished using the USPHS, NIH genetic sequence data bank. Alternatively, dsRNA containing appropriate siRNA sequences is ascertained using the strategy of Miyagishi and Taira (2003). DsRNA may be up to 800 base pairs long (Diallo M et al. 2003). The dsRNA optionally has a short hairpin structure (US Patent Application Publication 2004/0058886). Commercially available RNAi designer algorithms also exist (http://maidesigner.invitrogen.com/rnaiexpress/).

Ligand-RNA binding fusion proteins are prepared using existing plasmid technology (Caron et al. 2004; He et al. 2004). RNA binding proteins illustratively include histone (Jacobs and Imani 1988), RDE-4 (Tabara et al. 2002; Parrish and Fire 2001), and protamine (Warrant and Kim 1978). RNA binding protein cDNA is determined using the Gene Bank database (www.ncbi.nlm.nih.gov/IEB/Research/Acembly). For example, RDE-4 cDNA Gene Bank accession numbers are AY07926 and y1L832c2.3 (www.ncbi.nlm.nih.gov/IEB/Research/Acembly). RDE-4 initiates RNA interference by presenting dsRNA to Dicer (Tabara et al).

Alternatively, the RNA binding protein is covalently bound to a cell surface receptor specific ligand at the amino terminal of the ligand (Hermanson pp. 456-493).

Additional dsRNA binding proteins (and their Accession numbers in parenthesis) include: PKR (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, PO₅₇₉₇), and Dicer (BAA78691, AF408401, AAF56056, S44849, AAF03534, Q9884), RDE-4 (AY071926), FLJ20399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6ˆ (BAB83032, XP_{—}110167) CG1800 (AAF57175, EAA08039), FLJ20036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308 as enumerated in Saunders and Barber 2003.

Alternatively, cell surface receptor specific ligands that are rich in arginine and tyrosine residues are constructed such that those residues are positioned to form hydrogen bonds with engineered RNA containing appropriately positioned guanine and uracil (Jones 2001). Additionally, the necessity and performance of an internalization moiety is determined in vitro.

The suitability of the resulting ligand-dsRNA as a substrate for Dicer is first determined in vitro using recombinant Dicer (Zhang H 2002, Provost 2002, Myers J W 2003). Optimal ligand molecule size and dsRNA length are thereby identified.

In one embodiment, the ligand-dsRNA binding molecule(s) illustratively include: a histone (Jacobs and Imani 1988), RDE-4 (Tabara et al. 2002; Parrish and Fire 2001), and protamine (Warrant and Kim 1978) in order to render the ligand-dsRNA hydrophilic. The histone with relatively lower RNA-histone binding affinity (Jacobs and Imani 1988) such as histone H1 (prepared as described by Kratzmeier M et al. 2000) is preferred. Alternatively, RDE-4 is used as prepared commercially (Qiagen, Valencia, Calif.) using RDE-4 cDNA (Gene Bank accession numbers AY07926 and y1L832c2.3) (www.ncbi.nlm.nih.gov/IEB/Research/Acembly). RDE-4 initiates RNA interference by presenting dsRNA to Dicer (Tabara et al).

Protamines are arginine-rich proteins. For example, protamine 1 contains 10 arginine residues between amino acid residue number 21 and residue number 35 (RSRRRRRRSCQTRRR) (Lee et al. 1987) (SEQ ID NO. 15). Protamine binds to RNA (Warrant and Kim 1978).

Preparation of the ligand-histone-dsRNA complex is accomplished as described by (Yoshikawa et al. 2001). Complexes of ligand-lysine rich histone, the histone containing 24.7% (w/w) lysine and 1.9% arginine (w/w), with dsRNA is prepared by gentle dilution from a 2 M NaCl solution. Ligand-histone and dsRNA are dissolved in 2 M NaCl/10 mM Tris/HCl, pH 7.4, in which the charge ratio of dsRNA:histone (−/+) is adjusted to 1.0. Then the 2 M NaCl solution is slowly dispersed in distilled water in a glass vessel to obtain 0.2 M and 50 mM NaCl solutions. The final volume is 200 μL and final dsRNA concentration is 0.75 μM in nucleotide units.

Preparation of the ligand-RDE-4-dsRNA-complex is accomplished as described by (Johnston et al. 1992), for the conserved double-stranded RNA binding domain which RDE-4 contains. Ligand-RDE-4 binding to dsRNA to is accomplished in 50 mM NaCl/10 mM MgCl₂/10 mM Hepes, pH 8/0.1 mM EDTA/1 mM dithiothreitol/2.5% (wt/vol) non-fat dry milk.

Preparation of the ligand-protamine-dsRNA complex is accomplished as described by (Warrant and Kim 1978). The ligand-protamine (human recombinant protamine 1, Abnova Corporation, Taiwan, www.abnova.com.tw) and dsRNA at a molar ratio of 1:4 are placed in a buffered solution containing 40 mM Na cacodylate, 40 mM MgCl₂, 3 mM spermine HCl at pH 6.0 (Warrant and Kim 1978). The solution is incubated at 4° C.-6° C. for several days. Alternatively, the ligand-protamine-dsRNA complex is prepared as described by Song et al. 2005. The siRNA (300 nM) is mixed with the ligand-protamine protein at a molar ratio of 6:1 in phosphate buffered saline for 30 minutes at 4° C.

The constructed ligand-RNA binding protein-dsRNA complex is then administered parenterally and binds to its target cell via its receptor. The constructed ligand-RNA binding protein-dsRNA complex is then internalized and the dsRNA is hydrolyzed by Dicer thereby releasing siRNA for gene silencing.

### EXAMPLE 1

The Invitrogen Corporation (Carlsbad, Calif.) CellSensor CRE-bla Jurkat Cell-based Assay is used. The detailed protocol is available online and is included in the references (CellSensor protocol). Jurkat cells express CD38 on their cell surfaces which is internalized following ligand binding to it (Funaro at al. 1998). CellSensor CRE-bla Jurkat Cell-based Assay contains a beta-lactamase reporter gene under control of a cAMP response element which has been stably integrated into the CRE-b1a Jurkat cell line (clone E6-1). Beta-lactamase is expressed following forskolin stimulation.

Short interfering RNA 19 base pairs long is prepared using the Invitrogen Corporation algorithm based on the DNA sequence of the CRE-bla beta-lactamase gene:

(SEQ ID NO.16)

atggacccagaaacgctggtgaaagtaaaagatgctgaagatcagttggg
tgcacgagtgggttacatcgaactggatctcaacagcggtaagatccttg
agagttttcgccccgaagaacgttttccaatgatgagcacttttaaagtt
ctgctatgtggcgcggtattatcccgtattgacgccgggcaagagcaact
cggtcgccgcatacactattctcagaatgacttggttgagtactcaccag
tcacagaaaagcatcttacggatggcatgacagtaagagaattatgcagt
gctgccataaccatgagtgataacactgcggccaacttacttctgacaac
gatcggaggaccgaaggagctaaccgcttttttgcacaacatgggggatc
atgtaactcgccttgatcgttgggaaccggagctgaatgaagccatacca
aacgacgagcgtgacaccacgatgcctgtagcaatggcaacaacgttgcg
caaactattaactggcgaactacttactctagcttcccggcaacaattaa
tagactggatggaggcggataaagttgcaggaccacttctgcgctcggcc
cttccggctggctggtttattgctgataaatctggagccggtgagcgtgg
gtctcgcggtatcattgcagcactggggccagatggtaagccctcccgta
tcgtagttatctacacgacggggagtcaggcaactatggatgaacgaaat
agacagatcgctgagataggtgcctcactgattaagcattggtaa.

The DNA nucleotide sequence derived for suppressing beta-lactamase synthesis is: CCACGATGCCTGTAGCAAT (SEQ ID NO. 17). The complementary RNA oligonucleotide is prepared and annealed to its complementary strand sequences. This duplex siRNA is then incubated with anti-CD38 (Fab′)² fragment-histone (RNA binding protein) (Yoshikawa et al. 2001) or anti-CD38 (Fab′)² fragment-protamine (RNA binding protein) (Song et al. 2005). The siRNA-histone or protamine-anti-CD38 complex is incubated at 37° C. with the Jurkat cells for from 4 to 24 hours at concentrations ranging from 100 pM to 200 nM to evaluate efficacy. Typical efficacy is at 2 nM. Effective knockdown of intracellular synthesis of beta-lactamase is demonstrated in this system by the appearance of green cellular fluorescence. Positive control cells, which produce beta-lactamase, fluoresce blue.

### EXAMPLE 2

Multiple myeloma is a fatal incurable disease caused by the production of large amounts of a monoclonal immunoglobulin by malignant plasma cells (Grethlein S, Multiple Myeloma, eMedicine 2003). CD38 is a cell surface receptor found on myeloma plasma cells (Almeida J et al. 1999). Ligation of CD38 with anti-CD38 monoclonal antibodies (Serotec, Raleigh, N.C. and others) results in CD38 internalization (Pfister et al. 2001).

Anti-CD38 monoclonal antibodies are hydrolyzed by pepsin to produce anti-CD38 (Fab′)² fragments. Histone or protamine-anti CD38 (Fab′)² conjugate is prepared as described by Hermanson (Hermanson 1996, pp 456-493). The histone or protamine-anti-CD38 (Fab′)² conjugate is adsorbed to dsRNA containing a siRNA sequence that is complementary to a portion of the nucleotide sequence of the rearranged heavy chain of IgG (Yoshikawa et al. 2001, Song et al. 2005). In this case the nucleotide sequence link is X98954 and the GI number is 1495616. The siRNA sequences provided by the Whitehead Institute are:

| | | |
|---|---|---|
| S 5′: | CGCCAAGAACUUGGUCUAUUU | (SEQ ID NO. 18) |
| AS 3′: | UUGCGGUUCUUGAACCAGAUA. | (SEQ ID NO. 19) |

Alternatively, the histone or protamine-anti-CD38 (Fab′)² conjugate is adsorbed to the dsRNA containing a siRNA sequence that is complementary to a portion of the nucleotide sequence of the rearranged heavy chain of the IgG subclass of the subject's monoclonal IgG, i.e., IgG₁, IgG₂, IgG₃ or IgG₄.

The siRNA is then incorporated into dsRNA. Varying doses ranging from 0.4 to 15 grams of the histone or protamine-anti-CD38 (Fab′)² conjugate dsRNA are administered depending upon response. Effective doses of histone or protamine-anti-CD38 (Fab′)² conjugate dsRNA need to be administered at intervals ranging from one day to several days in order to maintain suppression of IgG production. Because the half life of IgG is up to approximately 23 days, the circulating concentration of the myeloma IgG will decrease gradually over several months. Suppression of the IgG subclass to which the IgG myeloma protein belongs will allow maintenance of IgG mediated immunity because the remaining IgG subclasses are not reduced. Improvement and/or prevention aspects of the disease which are consequences of high concentrations of the myeloma protein occur gradually as the concentration of the myeloma protein decreases. A direct effect of high concentrations of myeloma protein is hyperviscosity. This morbid effect of multiple myeloma is inhibited.

The histone or protamine-anti-CD38 (Fab′)² conjugate dsRNA containing the above described siRNA then binds to CD38 on the surfaces of the subject's plasma cells. Following internalization, Dicer hydrolyzes the dsRNA into siRNA which then interrupts the malignant plasma cell production of IgG myeloma protein.

### EXAMPLE 3

Allergic disease is mediated via IgE binding to the surfaces of mast cells and basophils. Upon bridging of adjacent IgE molecules by antigen, the mast cells and basophils are activated and release their mediators (Siraganian 1998). IgE binding by mast cells and basophils causes the signs and symptoms of allergic rhinitis, asthma, food and drug allergy, and anaphylaxis (e.g. Becker 2004). The amino acid sequence of the CH3 region of human IgE is available as are many of the codons (Kabat E A 1991). The DNA nucleotide sequence of the CH3 region of human IgE is readily deduced. The deduced CH3 region sequence is then provided to the Whitehead Institute's internet site as above to yield the corresponding siRNA sequence.

The histone or protamine-anti-CD38 (Fab′)² conjugate adsorbed to the anti-IgE siRNA then binds to CD38 on the surfaces of the subject's plasma cells. Following internalization, Dicer hydrolyzes the long dsRNA into siRNA which then interrupts the plasma cell production of the IgE. Over several months, the mast cell-bound and basophil-bound IgE is released and metabolized. The mast cell and basophil IgE receptors decrease markedly and the subject loses allergic reactivity.

### EXAMPLE 4

IgA nephropathy is an incurable disease of the kidney caused by deposition of IgA in the glomeruli of the kidneys (Brake M 2003). IgA₁ or IgA2 production is interrupted, depending upon the IgA subclass in the glomeruli, as described above for the silencing of IgG production. The progressive kidney damage caused by IgA is thereby interrupted.

### REFERENCES

Almeida J, Orfao A, Mateo G, Ocqueteau M, Garcia-Sanz R, Moro M J, Hernandez J, Ortega F, Borrego D, Barez A, Mejida M, San Miguel J F. Immunophenotypic and DNA content characteristics of plasma cells in multiple myeloma and monoclonal gammopathy of undetermined significance. Path Biol 1999; 47:119-127.
Anderson D C, Nichols E, Manger R, Woodle D, Barry M, Fritzberg A R. Tumor cell retention of antibody Fab fragments is enhanced by an attached HIV TAT protein-derived peptide. Biochem Biophys Res Commun 1993; 194:876-884.
Becker J M. Allergic Rhinitis, in In eMedicine, eds: Park C L, Mary L Windle M L, Georgitis J W, Pallares D, M D, Ballow M. 2004.
Brake M, Somers D. IgA Nephropathy in eMedicine, eds: Sondheimer J H, Talayera, F, Thomas C, Schmidt R J, Vecihi Batuman V. 2003.
Caron N J, Quenneville S P, Tremblay J P. Endosome disruption enhances functional nuclear delivery of Tat-fusion proteins. Biochem Biophys Res Commun 2004; 319:12-20.
CellSensor CRE-b1a Jurkat Cell-based Assay Protocol, Catalogue number K1134 (K1079), Invitrogen Corporation, Carlsbad, Calif.
Chiu Y-L, Ali A, Chu C-y, Cao H, Rana T M. Visualizing a correlation between siRNA localization, cellular uptake, and RNAi in living cells. Chem Biol 2004; 11:1165-1175.
Diallo M, Arenz C, Schmitz K, Sandhoff K, Scheppers U. Long endogenous dsRNAs can induce complete gene silencing in mammalian cells and primary cultures. Oligonucleotides 2003; 13:381-392.
Funaro A, Reinis M, Trubiani O, Santi S, Di Primio R, Malavasi F. CD38 functions are regulated through an internalization step. J Immunol 1998; 160:2238-2247.
Futaki S, Goto S, Sugiura Y. Membrane permeability commonly shared among arginine-rich peptides. J Mol Recognit 2003; 16:260-264.
Grethlein S. Multiple Myeloma. In eMedicine, eds: Krishnan K, Talayera F, Guthrie T H, McKenna Rajalaxmi, Besa E C 2003.
He D, Yang H, Lin Q, Huang H. Arg9-peptide facilitates the internalization of an anti-CEA imunotoxin and potentiates its specific cytotoxity to target cells. Int J Biochem Cell Biol 2005; 37:192-205.
Hermanson G T. Bioconjugate Techniques. Academic Press, San Diego, Calif. 1996.
Hutvagner G, Zamore P D. A microRNA in a multiple-turnover RNAi enzyme complex. Nature 2002; 297:2056-2060.
Hutvagner G, Zamore P D. RNAi: nature abhors a double-strand. Curr Opinion in Genetics and Development 2002; 12:225-232. Jacobs B L, Imani F. Histone proteins inhibit activation of the interferon-induced protein kinase by binding to double-stranded RNA. J Interferon Res 1988; 8:821-830.
Jo D, Nashabi A, Doxee C, Lin Q, Unutmaz D, Chen J, Ruley H E. Epigenetic regulation of gene structure and function with a cell-permeable Cre recombinase. Nature Biotechnology 2001; 19:929-933.
Jones S, Daley T A, Luscombe N M, Berman H M, Thornton J M. Protein-RNA interactions: a structural analysis. Nucl Acids Res 2001; 29:943-954.
Kabat E A, Wu T T, Perry H M, Gottesman K S, Foeller C. Sequences of Proteins of Immunological Interest. Fifth Edition. Tabulation and Analysis of Amino Acid and Nucleic Acid Sequences of Precursors, V-Regions, C-Regions, J-Chain, T-Cell Receptors for Antigen, T-Cell Surface Antigens, β₂-Microglobulins, Major Histocompatibility Antigens, Thy-1, Complement, C-Reactive Protein, Thymopoietin, Integrins, Post-gamma Globulin, a₂-Macroglobulins, and other Related Proteins. 1991. NIH Publication Number 91-3242.
Kratzmeier M, Albig W, Hanecke K, Doenecke D. Rapid dephosphorylation of HI histones after apoptosis induction. J Biol. Chem. 2000; 275:30478-30486.
Lee C-H, Hoyer-Fender S, Engel W. The nucleotide sequence of a human protamine 1 cDNA. Nucleic Acids Research 1987; 15:7639.
Mie M, Takahashi F, Funabashi H, Yanagida Y, Aizawa M, Kobatake E. Intracellular delivery of antibodies using TAT fusion protein A. Biochem Biophys Res Commun 2003; 310:730-734.
Miller V M, Xia H, Marrs G L, Gouvion C M, Lee G, Davidson B L, Paulson H L. Allele-specific silencing of dominant disease genes. Proc Natl Acad Sci USA 2003; 100:7195-7200.
Miyagishi M, Taira K. Strategies for generation of an siRNA expression library directed against the human genome. Oligonucleotides 2003; 13:325-333.
Muratovska A, Eccles M R. Conjugate for efficient delivery of short interfering RNA (siRNA) into mammalian cells. FEBS Letters 2004; 558:63-68.
Myers J W, Jones J T, Meyer T, Ferrell J E Jr. Recombinant Dicer efficiently converts large dsRNAs into siRNAs suitable for gene silencing. Nature Biotechnology 2003; 21:324-328.
Parrish S, Fire A. Distinct roles for RDE-1 and RDE-4 during RNA interference in Caenorhabditis elegans. RNA 2001; 7:1397-1402.
Peitz M, Pfannkuche K, Rajewsky K, Edenhofer F. Ability of the hydrophobic FGF and basic TAT peptides to promote cellular uptake of the recombinant Cre recombinase: A tool for efficient genetic engineering of mammalian genomes. Proc Natl Acad Sci USAS 2002; 99:4489-4494.
Pfister M, Ogilvie A, da Silva CP, Grahnert A, Guse A H, Hauschildt S. N AD degradation and regulation of CD38 expression by human monocytes/macrophages. Eur J Biochem 2001; 268:5601-5608.
Provost P, Dishart D, Doucer J, Frendewey D, Samuelsson B, Radmark O. Ribonuclease activity and RNA binding of recombinant human Dicer. EMBO J. 2002; 21:5864-5874.
St. Johnston D, Brown N H, Gall J G, Jantsch M. A conserved double-stranded RNA-binding domain. Proc Natl Acad Sci USA 1992; 89:10979-10983.
Saunders L A, Barber G N. The dsRNA binding protein family: critical roles, diverse cellular functions. FASEB J 2003; 17:961-983.
Siraganian R P. Biochemical events in basophil or mast cell activation and mediator release. Chapter 16 pp 204-227 in Allergy Principles and Practice, 5^{th} edition, eds E Middleton, Jr, C E Reed, E F Ellis, N F Adkinson, Jr, J W Yunginger W W Busse. Mosby, St. Louis, 1998.
Song E, Zhu P, Lee S-K, Chowdury D, Kussman S, Dykxhoom D M, Feng Y, Palliser D, Weiner D B, Shankar P, Marasco W A, Lieberman J. Antibody mediated in vivo delivery of small interfering RNAs via cell-surface receptors. Nature Biotechnology (epublication): 22 May 2005; doi:10.1038/nbt1101; (paper publication): 2005; 23:709-717.
Soomets U, Lindgren M, Gallet X, Hallbrink M, Elmquist A, Balaspiri L, Zorka M, Pooga M, Brasseur R, Langel U. Deletion analogues of transportan. Biochem Biophys Acta 2000; 1467:165-176.
Stura E A, Fieser G G, Wilson I A. Crystallization of antibodies and antibody-antigen complexes. Immunomethods 1993; 3:164-179.
Tabara H, Yigit E, Siomi H, Mello C C. The dsRNA binding protein RDE-4 interacts with RDE-1, DCR-1 and a DexH-Box helicase to direct RNAi in C. elegeans. Cell 2002; 109:861-871.
van Koningsbruggen S, de Haard H, de Kievit P, Dirks R W, van Remoortere A, Groot A J, van Engelen B G, den Dunnen J T, Verrips C T, Frants R R, van der Maarel S M. Llama-derived phage display antibodies in the dissection of the human disease oculopharyngeal muscular dystrophy. J Immunol Methods 2003; 279:149-161.
Warrant R W, Kim S-H. α-Helix-double helix interaction shown in the structure of a protamine-transfer RNA complex and a nucleoprotamine model. Nature 1978; 271:130-135.
Waterhouse P M, Wang M-B, Lough T. Gene silencing as an adaptive defense against viruses. Nature 2001; 411:834-842.
Yaneva J, Leuba S H, van Holde K, Zlatanova J. The major chromatin protein histone Hi binds preferentially to cis-platinum-damaged DNA. Proc Natl Acad Sci USA 1997; 94:13448-13451.
Yoshikawa Y, Velichko Y S, Ichiba Y, Yoshikawa K. Self-assembled pearling structure of long duplex DNA with histone H1. Eur J Biochem 2001; 268:2593-2599.
Zhang H, Kolb F A, Brondini V, Billy E, Filipowicz W. Human Dicer preferentially cleaves dsRNAs at their termini without a requirement for ATP. EMBO J. 2002; 21:5875-5885.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A composition comprising: a cell surface receptor specific ligand having a cell surface receptor specific binding site, said ligand conjugated to an RNA binding protein, said RNA binding protein adsorbed to a double-stranded RNA or to a small hairpin RNA sequence encoding a small interfering RNA operative to suppress production of a cellular protein.

2. The composition of claim 1 wherein said RNA binding protein is selected from the group consisting of: histone, protamine, RDE4 and PKR (Accession number in parenthesis) (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, PO₅₇₉₇), and Dicer (BAA78691, AF408401, AAF56056, S44849, AAF03534, Q9884), RDE-4 (AY071926), FLJ20399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6 (BAB83032, XP_{—}110167) CG1800 (AAF57175, EAA08039), FLJ20036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308.

3. The composition of claim 1 wherein said ligand is an immunoglobulin or an immunoglobulin fragment.

4. The composition of claim 3 wherein said immunoglobulin or immunoglobulin fragment is synthetic.

5. The composition of claim 1 wherein said bond extends from an amino terminus of said immunoglobulin or said immunoglobulin fragment to said RNA binding protein.

6. The composition of claim 1 wherein said ligand is a Fab immunoglobulin fragment.

7. The composition of claim 1 wherein said ligand is a (Fab′)² immunoglobulin fragment.

8. The composition of claim 1 wherein said double-stranded RNA is complementary to a cellular nucleotide sequence for a cell binding said ligand.

9. The composition of claim 1 wherein the ligand and RNA binding protein are conjugated in vitro.

10. The composition of claim 1 further comprising an internalization moiety having a bond to said ligand.

11. The composition of claim 1 wherein said internalization moiety has a bond to said RNA binding protein.

12. The composition of claim 10 wherein said internalization moiety is selected from the group of membrane-permeable arginine-rich peptides, pentratin, transportan, and transportan deletion analogs.

13. The composition of claim 1 wherein said ligand is an anti-CD38 (Fab′)² immunoglobulin fragment and said double-stranded RNA is complementary to a portion of a malignant cell genome.

14. The composition of claim 3 wherein said small interfering RNA sequence is complementary to an IgG heavy chain sequence.

15. The composition of claim 1 wherein said ligand is an anti-CD38 (Fab′)² immunoglobulin fragment and said double-stranded RNA is coding for an anti-immunoglobulin small interfering RNA.

16. The composition of claim 15 wherein said anti-immunoglobulin small interfering RNA is selected from the group consisting of: IgA, IgG, IgE, and IgM small interfering RNA.

17. A composition comprising:
a cell surface receptor specific ligand having a cell surface receptor specific binding site said ligand conjugated to an RNA binding protein conjugated to said ligand, said RNA binding protein adsorbed to a double-stranded RNA or to a small hairpin RNA sequence encoding a small interfering RNA operative to suppress production of a cellular protein and an internalization moiety having a bond to a compositional component selected from the group consisting of: said ligand and said RNA binding protein.

18. The composition of claim 17 wherein said RNA binding protein is selected from the group consisting of: histone, protamine, RDE4 and PKR (Accession number in parenthesis) (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, PO₅₇₉₇), and Dicer (BAA78691, AF408401, AAF56056, S44849, AAF03534, Q9884), RDE-4 (AY071926), FLJ20399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6 (BAB83032, XP_{—}110167) CG1800 (AAF57175, EAA08039), FLJ20036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308.

19. The composition of claim 17 wherein said internalization moiety is selected from the group of membrane-permeable arginine-rich peptides, pentratin, transportan, and transportan deletion analogs.

20. A composition comprising:
a fusion protein comprising: a cell surface receptor specific ligand having a cell surface receptor specific binding site;
an RNA binding protein combined with said ligand; and
an internalization moiety having a bond to a fusion protein component selected from the group consisting of: said ligand and said RNA binding protein; and
a double-stranded RNA encoding a small interfering RNA or a small hairpin RNA sequence operative to suppress production of a cellular protein adsorbed to said fusion protein.

21. The composition of claim 20 wherein said RNA binding protein is selected from the group consisting of: histone, protamine, RDE 4 and PKR (Accession number in parenthesis) (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, PO₅₇₉₇), and Dicer (BAA78691, AF408401, AAF56056, S44849, AAF03534, Q9884), RDE-4 (AY071926), FLJ20399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6 (BAB83032, XP_{—}110167) CG1800 (AAF57175, EAA08039), FLJ20036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308.

22. The composition of claim 20 wherein said immunoglobulin or immunoglobulin fragment is synthetic.

23. The composition of claim 20 wherein said bond extends from an amino terminus of said immunoglobulin to said RNA binding protein.

24. The composition of claim 20 wherein said ligand is a Fab immunoglobulin fragment.

25. The composition of claim 20 wherein said ligand is a (Fab′)² immunoglobulin fragment.

26. The composition of claim 20 wherein said double-stranded RNA is complementary to a cellular nucleotide sequence for a cell binding said ligand.

27. The composition of claim 20 wherein said internalization moiety is selected from the group of membrane-permeable arginine-rich peptides, pentratin, transportan, and transportan deletion analogs.

28. The composition of claim 20 wherein said ligand is an anti-CD38 (Fab′)² immunoglobulin fragment and said double-stranded RNA is complementary to a portion of a malignant cell genome.

29. The composition of claim 20 wherein said small interfering RNA sequence is complementary to an IgG heavy chain sequence.

30. The composition of claim 20 wherein said ligand is an anti-CD38 (Fab′)² immunoglobulin fragment and said double-stranded RNA is coding for an anti-immunoglobulin small interfering RNA.

31. The composition of claim 20 wherein said anti-immunoglobulin small interfering RNA is selected from the group consisting of: IgA, IgG, IgE, and IgM small interfering RNA.

32. The composition of claim 20 wherein said internalization moiety has a bond to said double-stranded RNA.

33. A process for suppressing cellular production of a protein comprising:
exposing a cell having a cell surface receptor to a composition of claim 1.

34. A process for suppressing cellular production of a protein comprising:
exposing a cell having a cell surface receptor to a composition of claim 20.


SEQUENCE LISTING (19 sequences)

SEQ ID NO: 1 - protein, 13 aa, Human immunodeficiency virus
  misc_feature: TAT 48-60
GRKKRRQRRRPPQ

SEQ ID NO: 2 - protein, 13 aa, Human immunodeficiency virus
  misc_feature: R9-Tat
GRRRRRRRRRPPQ

SEQ ID NO: 3 - protein, 17 aa, Human immunodeficiency virus
  misc_feature: HIV-1 Rev 34-50
TRQARRNRRRRWRERQR

SEQ ID NO: 4 - protein, 15 aa, flock house virus
  misc_feature: FHV coat 35-49
RRRRNRTRRNRRRVR

SEQ ID NO: 5 - protein, 19 aa, Brome mosaic virus
  misc_feature: gag 7-25
KMTRAQRRAAARRNRWTAR

SEQ ID NO: 6 - protein, 13 aa, Human T-cell lymphotropic virus type 2
  misc_feature: HTLV-II Rex 4-16
TRRQRTRRARRNR

SEQ ID NO: 7 - protein, 16 aa, Drosophilia atennapedia
  misc_feature: pentratin 43-58
RQIKIWFQNRRMKWKK

SEQ ID NO: 8 - protein, 26 aa, Unknown
  transportan
LIKKALAALAKLNIKLLYGASNLTWG

SEQ ID NO: 9 - protein, 27 aa, Unknown
  transportan
GWTLNSAGYLLGKINLKALAALAKKIL

SEQ ID NO: 10 - protein, 24 aa, Unknown
  transportan
LNSAGYLLGKINLKALAALAKKIL

SEQ ID NO: 11 - protein, 25 aa, Unknown
  transportan
GWTLNSAGYLLGKLKALAALAKKIL

SEQ ID NO: 12 - protein, 21 aa, Unknown
  transportan
AGYLLGKINLKALAALAKKIL

SEQ ID NO: 13 - protein, 22 aa, Unknown
  transportan
LNSAGYLLGKLKALAALAKKIL

SEQ ID NO: 14 - protein, 19 aa, Unknown
  transportan
AGYLLGKLKALAALAKKIL

SEQ ID NO: 15 - protein, 15 aa, Unknown
  transportan
RSRRRRRRSCQTRRR

SEQ ID NO: 16 - DNA, 795 bp, Unknown
  Cre-bla beta-lactamase
atggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 17 - DNA, 19 bp, Artificial
  Sequence derived for suppressing beta- lactamase expression
ccacgatgcctgtagcaat

SEQ ID NO: 18 - RNA, 21 nt, Artificial
  siRNA sequence complementary to portion of IgG heavy chain nucleotide sequence
cgccaagaacuuggucuauuu

SEQ ID NO: 19 - RNA, 21 nt, Artificial
  siRNA sequence complementary to portion of IgG heavy chain nucleotide sequence
uugcgguucuugaaccagaua